(19) United States
(12) Patent Application Publication
(10) Pub. No.: US 2025/0040810 A1
(43) Pub. Date: Feb. 6, 2025

(54) CREDIT, SECURITY, DEBIT CARDS AND THE LIKE WITH BUTTONS

(71) Applicant: DYNAMICS INC., Cheswick, PA (US)

(72) Inventors: Jeffrey David Mullen, Glenshaw, PA (US); Will Reutzel, Pittsburgh, PA (US)

(21) Appl. No.: 18/921,441

(22) Filed: Oct. 21, 2024

Related U.S. Application Data
(63) Continuation of application No. 12/339,092, filed on Dec. 19, 2008, now Pat. No. 12,121,328.
(60) Provisional application No. 61/016,491, filed on Dec. 24, 2007.
(60) Provisional application No. 61/026,846, filed on Feb. 7, 2008.
(60) Provisional application No. 61/027,807, filed on Feb. 11, 2008.
(60) Provisional application No. 61/081,003, filed on Jul. 15, 2008.
(60) Provisional application No. 61/086,239, filed on Aug. 5, 2008.
(60) Provisional application No. 61/090,423, filed on Aug. 20, 2008.
(60) Provisional application No. 61/097,401, filed on Sep. 16, 2008.
(60) Provisional application No. 61/112,766, filed on Nov. 9, 2008.
(60) Provisional application No. 61/117,186, filed on Nov. 23, 2008.
(60) Provisional application No. 61/119,366, filed on Dec. 2, 2008.
(60) Provisional application No. 61/120,813, filed on Dec. 8, 2008.

(51) Int. Cl. A61B 5/02; G06F 3/0488; G06K 7/00; G06K 7/08; G06K 7/10; G06K 19/06; G06K 19/07; G06K 19/073; G06K 19/077; G06K 19/08; G06Q 20/18; G06Q 20/20; G06Q 20/34; G06Q 20/38; G06Q 20/40; G06Q 30/0207; G06Q 30/0241; G06Q 30/0601; G06T 7/62; G06V 10/24; G06V 10/25; G07F 7/08; G07F 7/10

(52) U.S. Cl. CPC A61B 5/02042; A61B 5/02; G06F 3/0488; G06K 7/0004; G06K 7/084; G06K 7/087; G06K 7/10297; G06K 19/06187; G06K 19/06206; G06K 19/07; G06K 19/0702; G06K 19/0704; G06K 19/0723; G06K 19/0725; G06K 19/07345; G06K 19/07703; G06K 19/07705; G06K 19/07707; G06K 19/07709; G06K 19/07749; G06K 19/0775; G06K 19/07766; G06K 19/07769; G06K 19/07773; G06K 19/083; G06Q 20/18; G06Q 20/20; G06Q 20/34; G06Q 20/341; G06Q 20/3415; G06Q 20/352; G06Q 20/385; G06Q 20/401; G06Q 30/0222; G06Q 30/0241; G06Q 30/0277; G06Q 30/0641; G06T 7/62; G06V 10/24; G06V 10/25; G07F 7/0806; G07F 7/1008; G06T 2207/10024; G06T 2207/30004; G06V 2201/03

(57) ABSTRACT

A card is provided, such as a credit card or security card, that may transmit information to a magnetic stripe reader via a magnetic emulator. The emulator may transmit the information in order to reduce the amount of circuitry needed to emulate a particular block of information. Additionally, for example, one or more buttons may be included on the card. Buttons may be includes, for example, to provide a control interface to navigate through various options of the card. Additionally, coding schemes may be selected via buttons. Furthermore, a card may be locked until a private number is entered into a card or a number may only be generated (e.g., displayed and/or emulated) once a particular private number is entered into a card.

### CROSS-REFERENCE TO RELATED APPLICATIONS

This application claims the benefit of U.S. patent application Ser. No. 12/339,092 filed Dec. 19, 2008 (Docket No. D/004C), now U.S. Pat. No. 12,121,328 which issues on Oct. 22, 2024, Provisional Patent Application Nos. 61/016,491 filed on Dec. 24, 2007 (Docket No. JDM/019 PROV), 61/026,846 filed on Feb. 7, 2008 (Docket No. JDM/019PROV2), 61/027,807 filed on Feb. 11, 2008 (Docket. No. JDM/020 PROV), 61/081,003 filed on Jul. 15, 2008 (Docket No. D/005 PROV), 61/086,239 filed on Aug. 5, 2008 (Docket No. D/006 PROV), 61/090,423 filed on Aug. 20, 2008 (Docket No. D/007 PROV), 61/097,401 filed Sep. 16, 2008 (Docket No. D/008 PROV), 61/112,766 filed on Nov. 9, 2008 (Docket No. D/009 PROV), 61/117,186 filed on Nov. 23, 2008 (D/010 PROV), 61/119,366 filed on Dec. 2, 2008 (Docket No. D/011 PROV), and 61/120,813 filed on Dec. 8, 2008, all of which are hereby incorporated by reference herein in their entirety.

### BACKGROUND OF THE INVENTION

This invention relates to cards such as payment and security cards.

### SUMMARY OF THE INVENTION

A card is provided, such as a credit card or security card, that may transmit information to a magnetic stripe reader via a dynamic magnetic communications device such as a magnetic emulator or a magnetic encoder. The emulator may transmit the information serially, for example, in order to reduce the amount of circuitry needed to emulate a particular block of information (e.g., payment information).

One or more buttons may be included on the card. Buttons may be included, for example, to provide a control interface to navigate through various options of the card. Additionally, coding schemes may be selected via buttons. Furthermore, a card may be locked until a private number is entered into a card or a number may only be generated (e.g., displayed and/or emulated) once a particular private number is entered into a card. Such a number may be, for example, a dynamic credit card, security card, and/or debit card number or other number (e.g., security code).

A card, or other device, having a magnetic emulator may take the form of, for example, a credit card, debit card, and/or security card. Accordingly, the dynamic information may be a dynamic credit card number, a dynamic debit card number, and/or a dynamic security number. A display may be provided to display the data, or a portion of the data, communicated through an emulator. In this manner, a credit card may be provided that includes a display. All, or a portion of, a credit card number may, for example, be changed periodically and displayed on the display. Similarly, this changed information may be emulated via a parallel or serial emulator or other dynamic magnetic communications device (e.g., a magnetic encoder).

A dynamic magnetic communications device (e.g., magnetic emulators and/or encoders) may be located next to one or more magnetic stripe segments (e.g., sandwiched between two magnetic stripe segments from a birds-eye perspective of a card). A magnetic stripe may be utilized to transmit static information such that power is conserved. For example, if the beginning bits of a data block must take a particular form (e.g., start bits followed by user identification information) then this information may be embodied as a magnetic stripe. A serial or parallel emulator or encoder may then be provided to communicate the remaining information of the block (e.g., dynamic credit card number).

Numerous types of structures may be utilized to determine when a read-head of a magnetic stripe reader is reading, or is about to read, a magnetic stripe or dynamic magnetic communications device. Such structures may be utilized to turn a magnetic emulator, ON and OFF. By only turning an emulator ON when the emulator is in the proximity of a magnetic stripe reader, power may be conserved. For example, a button may be provided on a card, or other device, such that a user may provide manual input to instruct the card, or other device, to turn an emulator ON.

### BRIEF DESCRIPTION OF THE DRAWINGS

The principles and advantages of the present invention can be more clearly understood from the following detailed description considered in conjunction with the following drawings, in which the same reference numerals denote the same structural elements throughout, and in which:

FIG. 1 is an illustration of cards constructed in accordance with the principles of the present invention;

FIG. 2 is an illustration of cards constructed in accordance with the principles of the present invention;

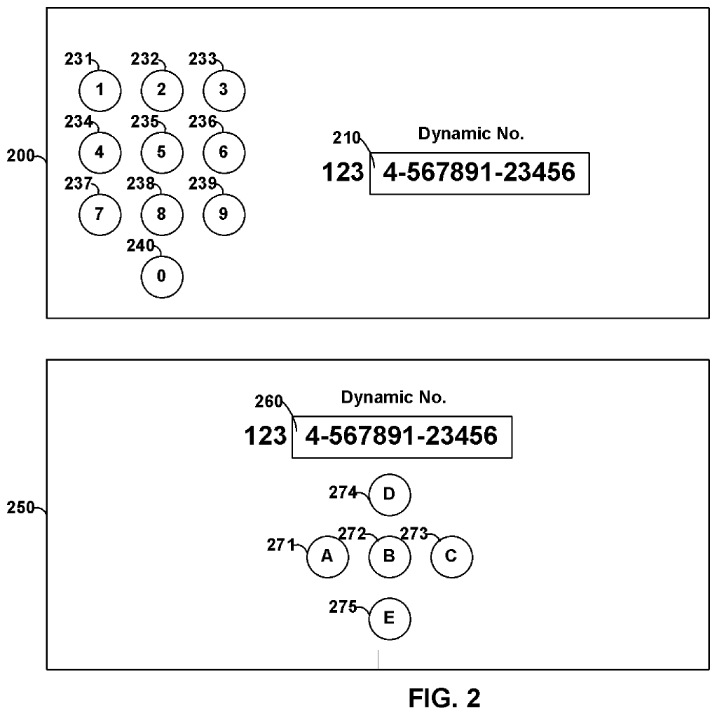

FIG. 3 is an illustration of cards constructed in accordance with the principles of the present invention;

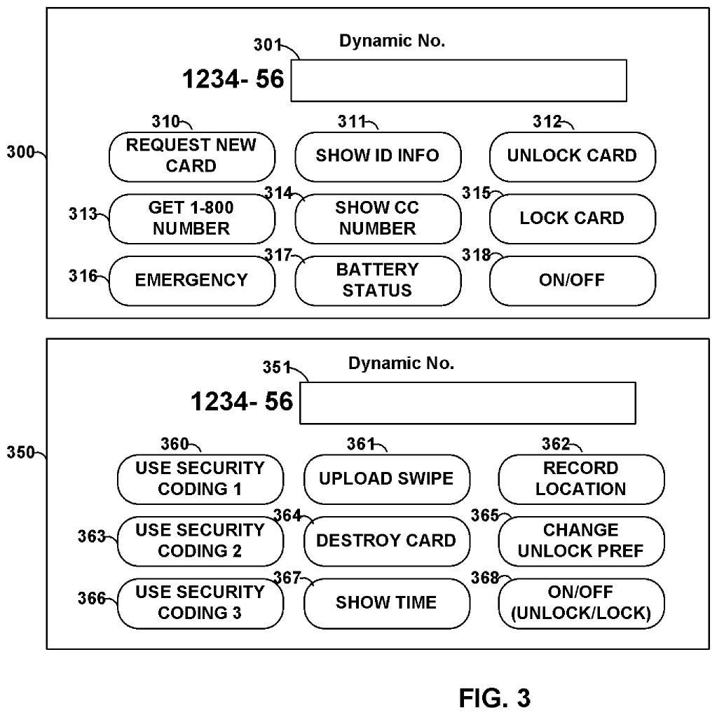

FIG. 4 is an illustration of cards constructed in accordance with the principles of the present invention;

FIG. 5 is an illustration of process topologies constructed in accordance with the principles of the present invention;

FIG. 6 is an illustration of cards constructed in accordance with the principles of the present invention;

FIG. 7 is an illustration of a card constructed in accordance with the principles of the present invention;

FIG. 8 is an illustration of a card constructed in accordance with the principles of the present invention; and

FIG. 9 is an illustration of a personal electronic device constructed in accordance with the principles of the present invention.

### DETAILED DESCRIPTION OF THE INVENTION

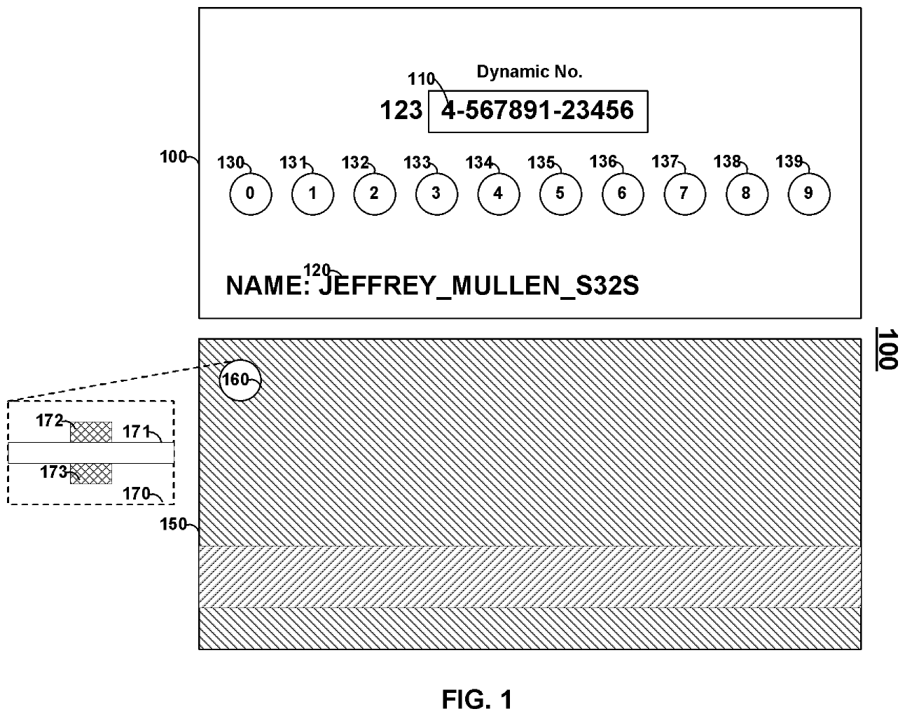

FIG. 1 shows card 100 that may include a display that may display dynamic number 110, which may be utilized, for example, as a credit card number or as part of a credit card number (e.g., with a static portion of a credit card number that proceeds dynamic number 110). Persons skilled in the art will appreciate that a dynamic number may take any forms such as, for example, a dynamic credit card number, a dynamic verification code number, and/or a dynamic security code number. For example, card 100 may include a dynamic credit card number and a dynamic verification code (e.g., a 15 digit credit card number and a 4 digit verification code or a 16 digit credit card number and a 3 digit code).

Identification information 120 may be provided on card 100. Accordingly, for example, a dynamic number may be provided for a particular period of time according to a coding scheme for that particular period of time. Thus, the identification information, time, and dynamic information may be transmitted via manual entry of that information (e.g., through a payment information input process on an online store) or via a magnetic emulator (e.g., through an in-store magnetic stripe reader). A remote server may receive such information and verify whether the dynamic information is correct for particular identification information and a particular period of time. A remote server may look at particular parts of a payment number (e.g., a static portion of a payment card number) and may determine whether another part of that payment number (e.g., a dynamic number) is valid for that particular part for a particular period of time. A number, or portion of a number, may be changed based on use (e.g., as a result of a user pressing a button or a read-head detector determining the presence of a magnetic stripe read-head).

Input buttons 130-139 may be provided such that manual input may be received and processed by card 100. Manual input buttons 130-139 may be utilized in a variety of ways. For example, an individual may be issued with a private personal identification number (PIN) to turn the card ON and/or to activate a feature. Thus, buttons 130-139 may be utilized to confirm that the individual issued the card is utilizing the card and its various features. In doing so with a credit card, for example, the amount of fraud associated with physical card theft may be minimized. Accordingly, a dynamic credit card number may be generated (e.g., coded) upon successful entry of an appropriate PIN. Additionally, for example, manual input keys 130-139 may be used to navigate through a list of options or initiate features. For example, button 130 may turn card 100 ON/OFF. Button 131 may turn display 110 ON/OFF. Button 132 may turn an emulator located on card 100 ON/OFF. Persons skilled in the art will appreciate that a credit card number may be coded based on time and transmitted with an identification number to a verification server. In turn, the verification server may decode the number based on time and identification number to verify, for example, a credit card transaction.

Card 150 may include button 160 which may, for example, be in the form of an aperture. For example the aperture may be defined in material 171 and may include sensors 172 to 173 to determine if a user presses around the aperture. Accordingly, a person pinching the aperture with two fingers may cause an electrical connection between sensors 172 and 173 via the skin of two fingers touching via the aperture. Accordingly, for example, pinching may result in the recognition of the activation of a button while just touching one side may not cause the activation of a button. In doing so, the number of times a button may become active by accident (e.g., while in a user's wallet) may be decreased.

Card 200 includes buttons 231-240. Buttons 231-140 may be aligned vertically or horizontally (e.g., with respect to the bottom of a card) or, for example, substantially in a block or circle.

Card 250 may include buttons 271-275, which may be aligned, for example, in the shape of a directional up-down/left-right pad with a centralized button. Accordingly, buttons 271-275 may be used to navigate through a list of options. Accordingly, for example, display 260 may include multiple lines of alphanumeric text and buttons 271-275 may be used to navigate through the test. Additionally, a personal identification code may be provided and may be entered via buttons 217-275 (e.g., ‘A-B-B-D-E-A’ may be entered to turn the card ON or turn a feature ON.

Card 300 may be included with buttons 310-319. Buttons 310-319 may also be associated with digits 0-8, respectively. Another button may be added and associated with, for example, digit 9 such that a digit-based keypad is provided. A digit may be pressed multiple times in succession such that alphanumeric data may be entered. Button 310 may be utilized to request a new card. In pressing button 310, or any button, information representative of this request may be displayed so that the information may be entered online or transmitted through a reader via a magnetic emulator. The receipt of such information may cause the desired action to occur (e.g., a new card may be sent).

Button 311 may be pressed to display and/or emulate identification information associated with the user of the card (or allow a user to LOGIN/LOGOUT of the card so that multiple users can utilize the card). Button 312 may be used to unlock the card. For example, button 312 may be pressed, then a personal identification code may be entered, then button 312 may be pressed again. If the correct personal identification code was entered, for example, then a feature (e.g., card unlocking) may occur. A process may, for example, include determining if button 312 is pressed and the entrance of a correct personal identification code without, for example, determining a subsequent entry of button 312. Such a process may, for example, allow a user to expedite entry of a personal identification code. If a user enters an incorrect personal identification code, for example, nothing may happen or the user may be prompted, via the display, to re-enter the code. After a particular amount of time waiting for the next manual input for a code, the processor may return to looking for the first manual input representative of a correct code (e.g., after 5 seconds). After an incorrect code is received, a processor may return to looking for the entry of the first manual input representative of a correct code (e.g., the first button of an appropriate code). Moreover, for example, a particular number of codes entered in error may permanently lock the card or may lock the card until a period of time has passed (e.g., 5 minutes). Button 313 may be added to present the 1800 number for the card on the display. Button 314 may be utilized to show, as well as magnetically emulate the a dynamic number (e.g., the dynamic credit card number for a period of time and for a particular person). Button 315 may be utilized for to lock a card. Button 316 may be utilized to transmit, for example, an emergency alert such as an alert that the card is about to be stolen or someone is in trouble (and may, for example, be transmitted upon swiping of a card). Button 317 may be utilized to display/magnetically emulate battery status. Button 318 may be utilized to turn the card ON/OFF. Persons skilled in the art will appreciate that, for example, additional information (e.g., alerts, battery information, card replacement requests) may be communicated as discretionary data in communicated payment information or may be communicated in a separate information transmission. Similarly, such information may be embedded in non-discretionary data information in communicated payment information.

Card 350 may be utilized. Buttons 360-368 may be provided. Buttons 360, 363, and 366 may each be associated with a different coding scheme. Pressing button 360, 363, and 366 may cause a number (e.g., credit card or security number) to be generated differently. Thus, for example, a company may issue security cards and may associate different button switch different levels of security or may rotate between the coding schemes or may allow for a new coding scheme to be used if a coding scheme is compromised. Button 361 may be utilized to upload information at upload locations (e.g., upload new software). Accordingly, circuitry may be included to receive information from a swipe.

Button 364 may be utilized to destroy a card (e.g., burn out components). Persons skilled in the art will appreciate that the functions of various buttons may be triggered autonomously upon particular determinations of a processor. For example, a processor may determine that someone is trying to break through the casing of a component (e.g., a memory) and may autonomously burn out components or perform other tasks (e.g., erase memory) as a result of the determinations. A processor may write information to a memory when the processor detects an fraudulent attack on a card by, for example, erasing a portion of data (e.g., payment card number(s)), erasing all of the data, or changing the data (e.g., replace a payment card number with a number indicative of a fraudulent activity) on a memory.

Button 367 may be utilized to show time (e.g., the current time) on a display. A clock may be provided on card 350 such that time may be kept. Such a clock may be provided with its own battery such that the clock may continue to keep track of time even when, for example, a processor is OFF. Persons skilled in the art will appreciate that a card may be ON when the card is delivered to a user but that a processor may be in a hibernation mode. Accordingly, for example, an ON/OFF button (or an unlock code) may wake that processor out of such a hibernation mode.

Button 363 may be utilized to record (e.g., store in memory), display on a display, and communicate through a dynamic magnetic communications device (e.g., a magnetic emulator or encoder) a location of the card or a history of locations of a card. Accordingly, for example, a locating device (e.g., GPS receiver) may be provided on a card. A transmitter may be provided that may communicate a signal that multiple remote receivers may receive (e.g., mobile phone base stations) such that the location of a card may be determined (e.g., via a triangulation process).

Button 365 may be utilized to change the unlocking preferences (e.g., change a personal identification code). For example, a user may be prompted to enter the user's current personal identification code, then be prompted to enter the user's new personal identification code, and then be prompted to confirm entry of the user's new personal identification code. If the two new personal identification codes match, then, for example, the personal identification code for a user may be changed. A card may be provided with a default personal identification code. Button 368 may be utilized on turn a card ON/OFF.

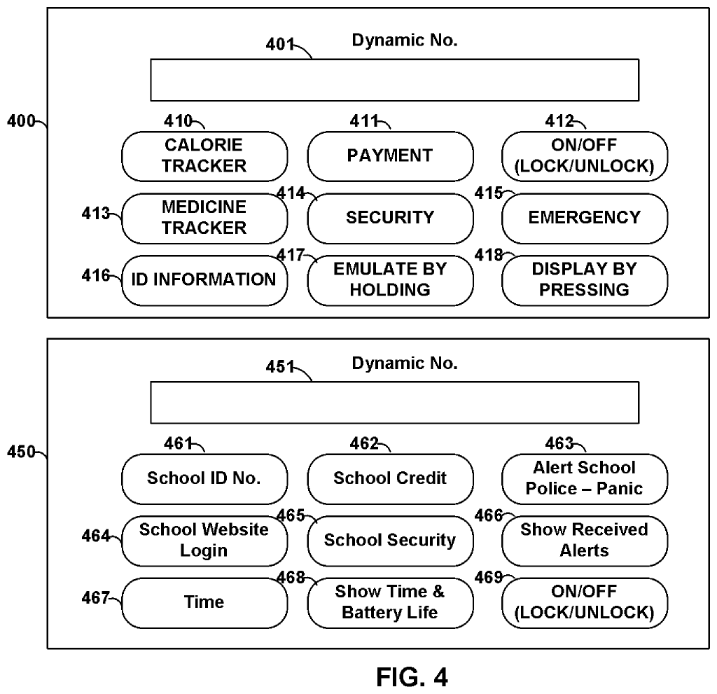

FIG. 4 shows card 400 which may include buttons 410-418. Button 410 may include a calorie tracker such that a user can enter in calories he/she eats per day. Thus, whenever a card is swiped via a magnetic stripe reader, or otherwise communicates data to a card reader or device, the calorie information may be entered into a database which can be utilized to populate a webpage (e.g., a calorie tracker webpage).

Button 413 may be utilized as a medicine tracker (e.g., to track the type and number of pills taken). Information may be displayed on display 401 that a user may enter such that the information may be associated with information entered by a user. For example, display 401 may provide an alphanumeric word “A342F2432S” that may be associated with, for example, 100 calories for breakfast and 300 calories for lunch on Dec. 12, 2007. This word may be entered on a website such that the information associated with the word may be used to populate the website (e.g., the calorie tracker).

Button 416 may be used to display identification information (e.g., name and phone number of card user). Accordingly, for example, someone that finds card 400 may press button 416 to determine the owner of card 416 as well as other information (e.g., phone number and email address).

Button 411 may be used for payment. Accordingly, for example, a payment number may be displayed on a display (along with additional payment data such as a payment security code). A dynamic magnetic communications device (or other device operable to communicate to a card reader) may also transmit information that includes such a payment number and additional payment data.

Button 414 may be used for security (e.g., an online login). Accordingly, for example, a user may press button 414 and may be provided with a code (e.g., an access security code) such that the user may enter particular portions of a website (e.g., a webpage associated with a user's banking account). Such an access code may be displayed to a user such that a user may enter the code into a keypad at a lock such that the lock is opened upon received of the correct code. Similarly, such an access security code may be, for example, communicated via a dynamic magnetic communications device (e.g., a magnetic emulator or magnetic encoder) as well as other reader communications devices (e.g., RFIDs and IC chips such as EMV chips). Such codes may change based on time or based on use (e.g., every time button 414 is pressed by a user).

Button 417 may be used to magnetically emulate information by holding button 416 such that data may be communicated via a magnetic emulator to a magnetic stripe reader. For example, button 410 may be pressed and then button 417 may be pressed to emulate information associated with a calorie tracker. Button 412 may be used, for example, to turn card 400 ON/OF and/or UNLOCK/LOCK card 400. Button 415 may be utilized as an emergency alert (e.g., a panic button). Accordingly, for example, a student may press emergency button 415 and swipe his/her card into a magnetic stripe reader and the appropriate authorities (e.g., police) may be alerted of the magnetic stripe reader, and its location, from which an emergency was initiated (and the identity of the person that initiated an emergency. In this manner, a police button, firefighter button, and ambulance button may be utilized.

Alternatively, for example, a doctor button, a nurse button, or a food button may be utilized for hospital cards. Button 418 may be utilized to display information on display 401 while the button is pressed. Accordingly, for example, calorie tracker 410 may be utilized and then button 418 may be pressed to display information associated with calorie tracker 410.

FIG. 4 shows card 450 that may include, for example, buttons 461-469. Button 461 may be used to display, as well as magnetically communicate via a magnetic emulator to a magnetic stripe reader, school identification information. Button 462 may be utilized to display, as well as communicate through a dynamic magnetic communications device, school credit information. Button 464 may be utilized to display, as well as communicate through a dynamic magnetic communications device, website login information. Button 467 may be utilized to display emulate, for example, time information. Button 466 may be utilized to show alerts that are received. For example, a receiver may be included in a card that may receive wireless alerts. Accordingly, for example, students may be alerted of a school-related risk/danger/information (e.g., bomb threat, fire, or school cancelled due to snow) and may be shown this information via display 451. Button 451 may be utilized to show, for example, the most recent alert and/or scroll through alerts. Button 469 may be utilized, for example, to turn card 450 ON/OFF and/or UNLOCK/LOCK card 450.

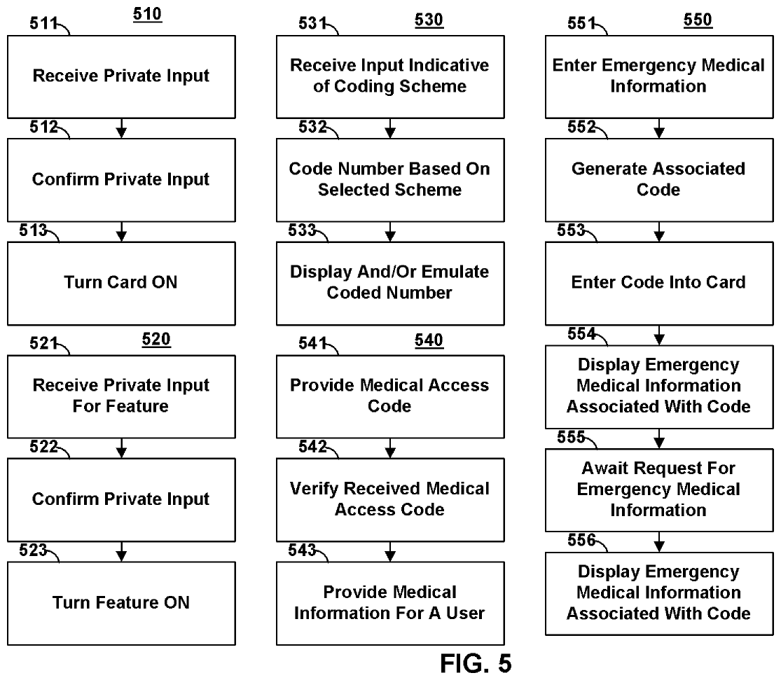

FIG. 5 shows flow charts 510, 520, and 530. Flow chart 510 may include, for example, step 511, in which private input (e.g., private identification information) is received. This information may be confirmed, for example, in step 512. Additionally, confirmation of the correct private number may turn a card ON (e.g., allow information to be displayed/emulated) in step 513.

Flow chart 520 may be included. Step 521 may be provided, in which, for example, private input is received for a particular feature. This input may be confirmed in step 522. Accordingly, for example, a feature may be turned ON in step 523. Flow chart 530 may be included. Step 531 may be provided, in which input indicative of a particular coding scheme is received. A number (e.g., website login and/or credit card) may be coded or generated (e.g., from a hash table associated with a particular input) in step 523. The coded, generated, and/or retrieved information may be displayed and/or communicated through a magnetic emulator in step 533.

Flow chart 540 may be provided and may be utilized, for example, in conjunction with a medical card and medical information retrieval system. For example, medical information may be stored on the memory of a card. Such medical information may be, for example, a user's height, weight, eye color, blood type, previous medical conditions, previous medications taken, current medical conditions, current medications taken, allergies, doctor contact information, as well as contact information for an emergency contact person.

Persons skilled in the art will appreciate that a user may control access to the user's medical information by, for example, keeping the medical information in his/her pocket and under his/her control at all times. (e.g., similar to the protection afforded to car keys and house keys). In the case of an emergency (e.g., a car accident), first responders may look for the user's medical card in order to gain access to the user's medical information. Such a medical card may take the form of, for example, an identification card (e.g., a driver's license or passport). A sticker may be placed on a card or device (e.g., a mobile telephone or identification card) stating that a user has a medical card in his/her wallet (e.g., as well as the location of the card such as on the left-hand side of the wallet). A medical card may, for example, be taken by a first responder and may display a passcode for the responder to enter onto a website in order for the responder to obtain the user's medical information. Identification information may be permanently displayed on the card (e.g., printed or embossed) and this identification may be entered into a website along with a user. Instructions for accessing the medical information may be printed or embossed on a card or other device. Such an access security code may, for example, change based on time or use (e.g., press of a particular button or particular buttons). A first responder may be prompted by a website, for example, to enter in a responder's username and password such that the responder can be identified as a responder that may access the medical information of a user. Medical information stored on a remote server may include, for example, pictures (e.g., of a birth certificate and bodily parts at various times), x-rays, medical reports, as well as any other type of medical information. A medical card may also store such images and other data.

Flow chart 540 may include, for example, a card (or other device) providing a medical access code in step 541. Step 542 may be included, in which a medical access code is verified (e.g., on a remote server). Step 543 may be provided, in which medical information for a user may be provided as the result of the verification of a correct access code. An access code may be, for example, a five, six, seven, or eight digit code (e.g., “834699”). Persons skilled in the art will appreciate that a card may include, for example, medical information. Such medical information may be displayed, for example, by a user pressing a particular button. The information may be scrolled left/right as well as up/down using the same button or additional buttons. For example, a first line of data may be “Blood Type: B” and a second line of data that can be scrolled down to using a button may be “Allergies: None”

Flow chart 550 may be provided. Step 551 may be provided, in which a user may go to a website or a graphical user interface on a device and enter in his/her emergency medical information. Such information may be, for example, pre-populated with the websites prior knowledge as to the user's emergency medical information. Such information may be changed by a user. The entry of medical information may take many forms. For example, the entry of medical information may be done through the selection of options. For example, a user may be provided with a list of allergies and may select those allergies that apply to a user. A user may then, for example, generate a code in step 552. Such a code may be, for example, associated with the particular combination of selections that user made. A user may then, for example, enter this code into his/her medical card using buttons on that medical card. In this manner, the medical card may include data on a memory that may recognize the code and may display, at a user's request, the medical information associated with that code (e.g., step 554). Accordingly, for example, a user may customize and update his/her payment card without having to connect the user's payment card to a computer (e.g., via a USB port). A card may wait for a request for emergency medical information (e.g., step 555) and may provide the emergency medical information as a result of receiving the request (e.g., step 556).

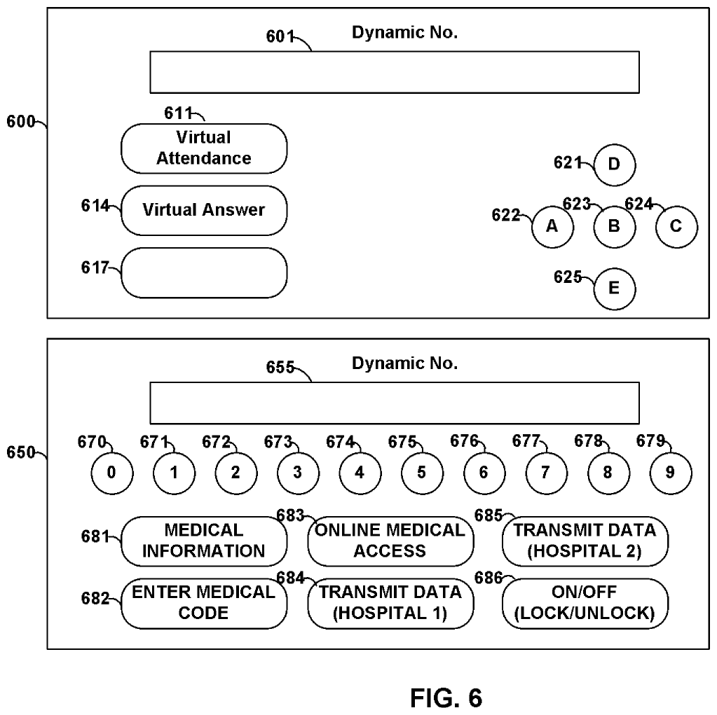

FIG. 6 shows card 600 which may include buttons 611, 614, and 617. Button 611 may be utilized for virtual attendance. A user may press button 611 and transmit identification information (e.g., either wirelessly or via a magnetic emulator) to a server such that attendance may be recorded. Similarly, button 614 may be utilized to provide a virtual answer to a question. For example, button 614 may be pressed, a button associated with answer “B” may be pressed, button 614 may be pressed again, and then a card may be swiped and information associated with the answer transmitted (e.g., via a magnetic emulator) to a server for further processing. Buttons 621-625 may be utilized, for example, to enter responses into a card so that the responses may be displayed visually or communicated via a magnetic emulator.

Card 650 may be provided with buttons 670-679 and 681-686. Button 681 may be utilized, for example, to display medical information on a display of card 650. Button 682 may be utilized, for example, to prompt a processor on card 650 that a code associated with medical information is about to be entered. Button 683 may be utilized, for example, to provide (e.g., via a display) a code for accessing a user's online medical record. Button 684 may be utilized, for example, to communicate information (e.g., insurance information) in one format to a particular hospital that accepts that format. Button 685 may be utilized, for example, to communicate the same information (e.g., the same insurance information) in a different format to a different hospital that accepts that different format. Button 686 may be utilized to turn a card ON/OFF.

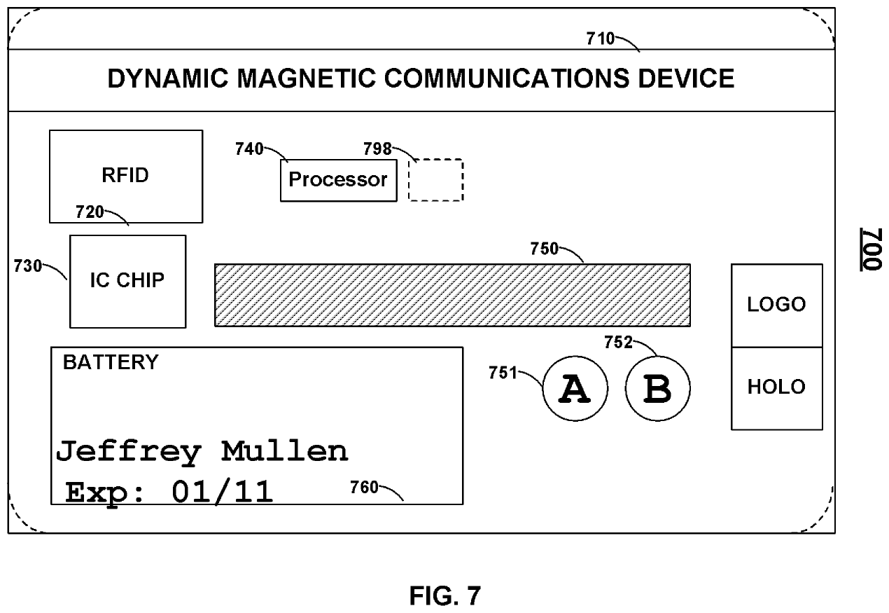

FIG. 7 shows card 700 that may include, for example, one or more IC chips 730 (e.g., EMV chips), RFID antennas 720, processors 740, displays 750, dynamic magnetic communications devices 810 (e.g., magnetic encoders and/or magnetic emulators), batteries 760, and buttons 751 and 752. Additional circuitry 798 may be provided which may be, for example, one or more oscillators or emulator driving circuits. Persons skilled in the art will appreciate that button 751 may, for example, be utilized by a user to select one encryption algorithm for a number displayed on display 750 while button 752 may be utilized by a user to select a different encryption algorithm. Persons skilled in the art will appreciate that the components of card 700 may be provided on either surface of a card (e.g., a front or rear surface of the card) or inside of a card. A logo (e.g., of a card issuer) and logo may be provided on either surface of a card.

A button, such as button 751, may be utilized, for example, to display a number. Such a number may be, for example, encrypted from a secure number based on time or use. For example, one-time use numbers (e.g., a payment number or code) may be retrieved from a list of numbers on memory each time button 751 is pressed and displayed on display 750. A processor may only go through each number once on a list. A registration process may be provided in which a user may be requested to enter in a sequence of numbers such that a remote server may validate the card and learn where in a sequence of a list a card currently resides. Numbers may be repeated on a list or may only occur once on a list. All of the numbers available by the length of the number may be utilized by the list or only a portion of the numbers available by the length of the number may be provided by the list. A secret number may be encrypted on a card and a verification server may also have knowledge of this secret number. Accordingly, the remote server may perform the same encryption function as the card on the secret number and verify that the resultant encrypted number is the same as the resultant encrypted number on a card. Alternatively, for example, the remote server may decrypt the received encrypted number to determine the authenticity of the encrypted number and validate an activity (e.g., validate a security access request or a purchase transaction).

Persons skilled in the art will appreciate, for example, that a card may include an IC chip (e.g., EMV chip), RFID, and a dynamic magnetic communications device (e.g., a magnetic emulator or encoder). The same information may be communicated through, for example, any number of such devices (e.g., a dynamic magnetic communications device, RFID, and an EMV chip). A central processor may cause each device to communicate the information (in the same format or a different format). Each component may have its own processor or driving circuitry. Such individual processors or driving circuitry may be coupled to a central processor. An EMV chip may be utilized, for example, to provide control signals to other devices (e.g., circuitry driving a display as well as a dynamic magnetic communications device). Such an EMV chip may receive signals provided by one or more buttons to determine, for example, that a particular button, or sequence of buttons, was pressed by a user.

Persons skilled in the art will appreciate that a read-head housing may include, for example, multiple read-heads. A read-head detector may, more generally, detect a read-head housing and, in doing so, detect a read-head.

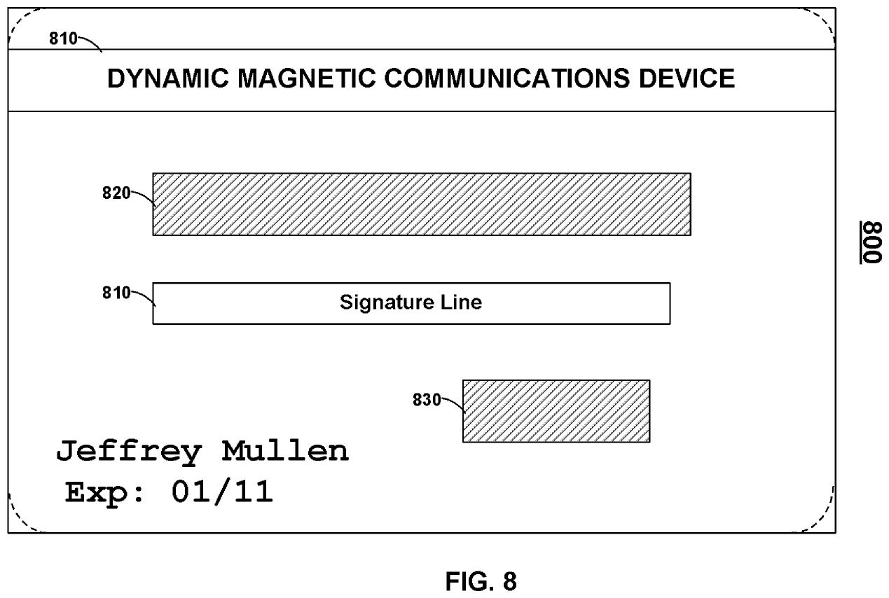

FIG. 8 shows card 800 that may include, for example, signature area 810 that may include a material operable to receive marks from a pen (e.g., a signature). Card 800 may also include, for example, displays 820 and 830. Display 820 may, for example, display a payment number while display 830 displays a security code (e.g., for online purchase authentication). Display 820 as well as display 830 may be utilized on the same side as, for example, dynamic magnetic communications device 810.

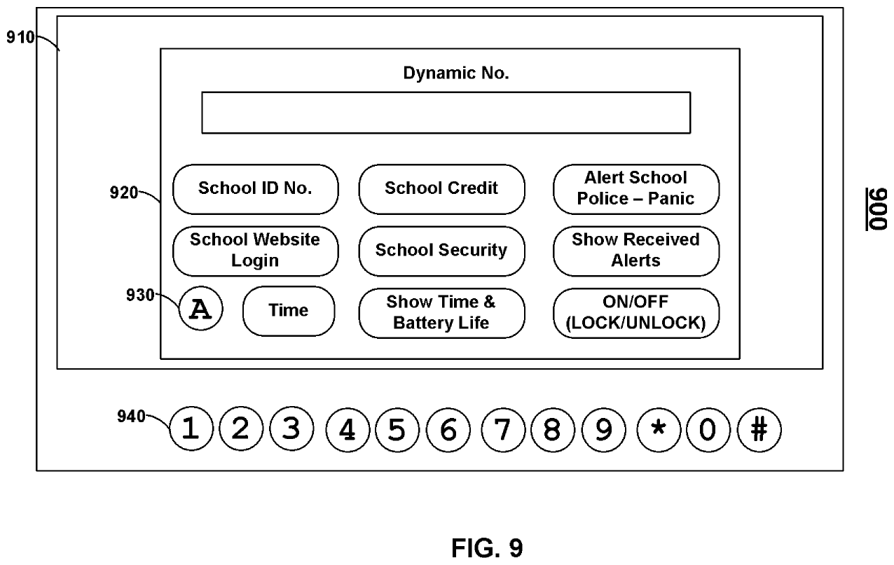

FIG. 9 shows personal electronic device 900 which may be, for example, a portable telephonic device, portable media player, or any type of electronic device. Persons skilled in the art will appreciate that the functionality of a card may be provided on a personal device and displayed through a graphical user interface. Personal electronic device 900 may include, for example, user inputs 940 and display 910. Virtual card 920 may be displayed on display 920. Display 920 may be a touch-sensitive display such that, for example, virtual button 930 may be provided on virtual card 920. Persons skilled in the art will appreciate that cards may be provided as virtual cards and a user may interact with such virtual cards in order to provide a variety of functions. Personal electronic device 900 may communicate to a card reader such as, for example, an RFID reader.

A display may be bi-stable or non bi-stable. A bi-stable display may consume electrical energy to change the information displayed on the bi-stable display but may not consume electrical energy to maintain the display of that information. A non bi-stable display may consume electrical energy to both change and maintain information on the non bi-stable display. A display driving circuit may be provided, for example, for a bi-stable display (or a non bi-stable display). Such a display driving circuit may step-up a supply voltage (e.g., 1-5 volts) to a larger voltage (e.g., 6-15 volts) such that a bi-stable display may change displayed information. A controller (e.g., a processor) may be utilized to control such a display driving circuit. Persons skilled in the art will appreciate that a display may be configured to display numerical data or alphanumerical data. A display may also be configured to display other indicia (e.g., the image of a battery and its remaining life).

Persons skilled in the art will appreciate that a dynamic magnetic communications device (e.g., a magnetic emulator or magnetic encoder) may be fabricated, either completely or partially, in silicon and provided as a silicon-based chip. Other circuitry (e.g., driving circuitry) may also be fabricated on such a silicon-based chip. A processor, such as a processor for controlling a magnetic communications device, may be, for example, a programmable processor having on-board programmable non-volatile memory (e.g., FLASH memory), volatile memory (e.g., RAM), as well as a cache. Firmware as well as payment information (e.g., dynamic numbers) may be, for example, communicated from a programming device to a processor's on-board programmable non-volatile memory (e.g., a FLASH memory) such that a card may provide a variety of functionalities. Such a processor may also have one or more power-saving operating modes, in which each operating mode turns OFF a different set of circuitry to provide different levels of power consumption. One or more power-savings modes may turn OFF, for example, one or more clocking circuitry provided on a processor. An Application-Specific Integrated Circuit (ASIC) may also be included in a card or other device to provide, for example, processing, dynamic magnetic communications, as well as driving capabilities.

Persons skilled in the art will also appreciate that the present invention is not limited to only the embodiments described. Instead, the present invention more generally involves dynamic information. Persons skilled in the art will also appreciate that the apparatus of the present invention may be implemented in other ways then those described herein. All such modifications are within the scope of the present invention, which is limited only by the claims that follow.

What is claimed is:

1. A card comprising:
a plurality of buttons;
a dynamic magnetic communications device operable to communicate at least one track of magnetic stripe information to a read head of a magnetic stripe reader of a payment terminal, the dynamics communication device being fabricated on a first printed circuit board;
RFID circuitry operable to communicate with RFID circuitry of a payment terminal;
processing circuitry that generates a dynamic credit card number upon successful entry of a stored PIN number; and
a display that displays said dynamics credit card number, wherein said card is operable to transmit said dynamic credit card number to said payment terminal through at least one of said dynamic magnetic communications device and said RFID circuitry.

2. The card of claim 1, wherein said display is non bi-stable.

3. The card of claim 1, wherein said display is bi-stable.

4. The card of claim 1, wherein said display is located above said plurality of buttons.

5. The card of claim 1, wherein said display is located to the right of said plurality of buttons.

6. The card of claim 1, further comprising a battery.

7. The card of claim 1, further comprising a card reader communications device.

8. The card of claim 1, further comprising magnetic stripe read-head detector circuitry.

9. The card of claim 1, further comprising a card reader communications device for communicating a data block.

10. The card of claim 1, further comprising a card reader communications device for communicating a data block upon successful entry of a stored PIN number.

11. The card of claim 1, further comprising a card reader communications device for communicating a data block serially.

12. The card of claim 1, successful entry of a stored PIN number comprises circuitry that matches a sequence of button entries with a stored personal identification number.

13. The card of claim 1, wherein data transmitted to said payment terminal comprises at least a portion of a payment number.

14. The card of claim 1, wherein data transmitted to said payment terminal comprises at least a portion of a credit card number.

15. The card of claim 1, wherein data transmitted to said payment terminal includes at least a portion of a debit card number.
